(19) United States
(12) Patent Application Publication
(10) Pub. No.: US 2011/0124003 A1
(43) Pub. Date: May 26, 2011

(54) Cytological Methods for Detecting Cancer

(75) Inventor: David Allen Ralph, Edmond, OK (US)

(73) Assignee: INTEGRATED BIOSCIENCE SOLUTIONS, LLC, Cincinnati, OH (US)

(21) Appl. No.: 12/989,325

(22) PCT Filed: Apr. 24, 2009
(86) PCT No.: PCT/US2009/041642

§ 371 Date: Jan. 10, 2011

Related U.S. Application Data
(60) Provisional application No. 61/048,055, filed on Apr. 25, 2008.

(51) Int. Cl. C12Q 1/68; G01N 33/53

(52) U.S. Cl. 435/6.12; 435/7.21; 435/6.14

(57) ABSTRACT

The present invention relates to methods for diagnostics, detection or research analysis of cancer. In particular, the present invention is in the field of analysis of the levels of gene expression in normal or noncancerous cells because of their prosximity to cancer cells. The present invention further provides for analysis of the altered gene expression levels in normal or non-cancerous cancerous cells as an indicator of disease prognosis, staging and grading. The current invention is a means to increase the sensitivity of needle core biopsies to detect the presence of cancer.

### CROSS-REFERENCE TO RELATED APPLICATIONS

This application claims the benefit of U.S. Provisional Patent Application Ser. No. 61/048,055, filed Apr. 25, 2008, and PCT Application Serial No. PCT/US09/41642, filed Apr. 24, 2008, both entitled “Examination Of Normal Or Noncancerous Cells In Cytology Specimens To Detect Cancer,” incorporated herein by reference for all purposes.

### FIELD OF INVENTION

The present invention relates to methods for diagnostics, detection or research analysis of cancer. In one embodiment, the present invention is in the field of analysis of the levels of gene expression in normal or noncancerous cells because of their proximity to cancer cells. The present invention further provides for analysis of the altered gene expression levels in normal or noncancerous cells as an indicator of disease presence, positioning, prognosis, staging and grading.

### BACKGROUND OF THE INVENTION

Many cellular events and processes are characterized by altered expression levels of one or more genes. Differences in gene expression patterns correlate with changes in disease or physiological state.

Prostate cancer is usually diagnosed by pathologists examining needle core biopsies collected from patients by urologists. There is a deficiency in the current art whereby biopsy sampling is insufficient to detect all prostate cancer tumors. In current practice, a urologist may collect six to twelve and possibly more needle core biopsies from a single patient in the hope of having at least one of the biopsy cores sample a patient's tumor.

Unfortunately, even when many needle core biopsies are obtained from a single patient who has a prostate tumor, there is still a significant possibility that none of the biopsies will sample tissue from the tumor. Without a sampling of the prostate tumor, a pathologist cannot diagnose the cancer. At the very least, this failure to diagnose an existing cancer delays providing patients with potentially life saving cancer treatments. Stated differently, the current practice of diagnosing prostate tumors with needle core biopsies lacks the desired sensitivity to detect prostate cancer. Urologists understand this situation and frequently repeat a patient's biopsy in order to detect cancers that were missed in the first biopsy attempt. Certainly, repeating biopsies results in identification of more but not all cancers missed on the first biopsy attempts, however; the practice of repeat biopsies also creates a situation in which men without cancer are repeatedly biopsied.

The current invention is a means to increase the sensitivity of needle core biopsies to detect the presence of prostate cancer. Those skilled in the art will recognize that the current invention may have applications supporting the diagnosis of other cancers. This disclosure will also discuss possible application of the current invention to the detection of cancers of the bladder and breast. This is not intended to be an exhaustive list of the possible applications of the current invention. There are certainly other applications of the current invention for the detection of cancer.

### BRIEF SUMMARY OF THE INVENTION

The present invention relates to methods for diagnostics, detection or research analysis of cancer. In one embodiment, the present invention is in the field of analysis of the levels of gene expression in normal or noncancerous cells because of their proximity to cancer cells. The present invention further provides for analysis of the altered gene expression levels in normal or noncancerous cells as an indicator of disease presence, positioning, prognosis, staging and grading.

According to one embodiment of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein differential expression of the genes is indicative of cancer.

In another aspect of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein increased expression of the genes that are up-regulated is indicative of cancer.

In another aspect of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein increased expression of the genes that are down-regulated is indicative of cancer.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein the differential expression of the genes is indicative of the cancer progression.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein increased expression of the genes that are down-regulated is indicative of cancer progression.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein increased expression of the genes that are up-regulated is indicative of cancer progression.

The invention also includes methods of differentiating metastatic cancer from nonmetastatic cancer in a patient comprising the step of detecting the level of expression in anatomically normal or noncancerous cells in a cytology specimen consisting of cells that had previously resided in the proximity of cancer; wherein altered patterns of gene expression is indicative of metastatic cancer rather than nonmetastatic cancer.

In another aspect of the invention, a method is provided for diagnosing the presence or absence of cancer in a patient can be used to detect, for example, prostate cancer, bladder cancer, liver cancer, lung cancer, and breast cancer, just to name a few examples.

In one embodiment of the present invention the tumor is a gastrointestinal tumor, for example a tumor of the esophagus, the stomach, the pancreas, the bile tree, the liver, the small intestine, the colon or the rectum. In another embodiment the tumor is for example cancer of the esophagus, gastric cancer, cancer of the gallbladder, the pancreas, the liver, the small intestine, the colon or the rectum. The tumors according to the present invention may comprise tumors, which show detectable lymph-node involvement (node positive tumors) as well as tumors, without detectable spread to lymph nodes (node negative tumors). In one preferred embodiment of the invention the gastrointestinal tumors are tumors without detectable spread to lymph nodes.

Poor clinical outcome can be measured, for example, in terms of shortened survival or increased risk of cancer recurrence, e.g. following surgical removal of the cancer.

In another embodiment, the invention concerns a method of predicting the likelihood of the recurrence of cancer, following treatment, in a cancer patient, comprising determining the expression level of certain genes, or its expression product, in a cancer tissue obtained from the patient, normalized against a control gene or genes, and compared to the amount found in a reference cancer tissue set, wherein an expression level indicates decreased risk of recurrence following treatment.

All types of cancer are included, such as, for example, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer. The foregoing methods are particularly suitable for prognosis/classification of breast cancer.

In all previous aspects, in a specific embodiment, the expression level is determined using RNA obtained from a formalin-fixed, paraffin-embedded tissue sample. While all techniques of gene expression profiling, as well as proteomics techniques, are suitable for use in performing the foregoing aspects of the invention, the gene expression levels are often determined by reverse transcription polymerase chain reaction (RT-PCR).

The expression data can be further subjected to multivariate analysis, for example using the Cox Proportional Hazards model.

The invention further includes computer systems comprising a database containing information identifying the expression level in an identified tissue of a set of genes; and a user interface to view the information. The database may further include sequence information for the genes, information identifying the expression level for the set of genes in normal tissue and malignant tissue (metastatic and nonmetastatic) and may contain links to external databases such as GenBank.

Lastly, the invention includes methods of using the databases, such as methods of using the disclosed computer systems to present information identifying the expression level in a tissue or cell, comprising the step of comparing the expression level in the tissue or cell to the level of expression of the gene in the database.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

Throughout this document, all temperatures are given in degrees Celsius, and all percentages are weight percentages unless otherwise stated. All publications mentioned herein are incorporated herein by reference in their entirety for all purposes, which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

### DETAILED DESCRIPTION OF THE INVENTION

Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “differentially expressed gene,” “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes, or a comparison of the ratios of the expression between two or more genes, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is a measurable and statistically significant change in the abundance of a gene's mRNA and/or encoded protein product relative to another gene's or genes' mRNA(s) and /or encoded protein(s) abundance. A gene is differentially expressed if the abundance of its mRNA and/or its encoded protein changes relative to the abundance of one other gene's mRNA and/or protein. Alternatively, a gene is differentially expressed if its mRNA and/or encoded protein abundance changes relative to the abundances of the mRNAs and/or encoded proteins derived from two or more and possible all other genes.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, may also increase in the proportion of the number of copies made of the particular gene expressed.

The term “prognosis” is used herein to refer to the prediction of the likelihood of cancer-attributable death or progression, including recurrence, metastatic spread, and drug resistance, of a neoplastic disease, such as breast cancer. The term “prediction” is used herein to refer to the likelihood that a patient will respond either favorably or unfavorably to a drug or set of drugs, and also the extent of those responses. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The term “tumor,” as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth. Examples of cancer include but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

A cytology specimen is a sampling of cells from an organ, tissue, bodily fluid or other region of the body. A key feature of a cytology specimen is that the cells in such a cytology specimen are disaggregated and occur singly or in small clusters. Information concerning the anatomical context of exactly where in the organ, tissue, bodily fluid or region of the body from which a cell in a cytology specimen was obtained cannot be directly observed. Commonly, cytology specimens are collected as suspensions of cells and/or small clusters of cells in a fluid medium of some kind. The cells and cell clusters in a cytology specimen can be collected or concentrated by centrifugation or filtration. As used in the context of the current invention, a cytology specimen can mean the cellular suspensions and/or their simple derivatives created by centrifugation or filtration.

A “cytology sample” encompasses any sample obtained from a living system or subject. The definition encompasses blood, serum, tissue, and other samples of biological origin that can be collected from a living system, subject or individual. In one embodiment, biological samples are obtained through sampling by minimally invasive or non-invasive approaches (e.g., urine collection, stool collection, blood drawing, needle aspiration, and other procedures involving minimal risk, discomfort or effort). Cytology samples are often liquid (sometimes referred to as a “biological fluid”). Liquid cytology samples include, but are not limited to, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, and others. Cytology samples include samples that have been manipulated in any way after their procurement, such as by treatment with reagents, various fixatives, solubilization, or enrichment for certain components, such as proteins or polynucleotides. The term “cytology sample” also encompasses a clinical sample such as serum, plasma, other biological fluid, or tissue samples, and also includes cells in culture, cell supernatants and cell lysates.

As used in the context of the current invention, normal or noncancerous means anatomically normal or noncancerous appearing when viewed with the aid of a microscope. Cells that appear normal or noncancerous may have altered patterns of gene expression and/or altered physiologies that may be indicative of or associated with a pathological state.

The present invention relates to methods for diagnostics, detection or research analysis of cancer. In one embodiment, the present invention is in the field of analysis of the levels of gene expression in normal or noncancerous cells because of their proximity to cancer cells. The present invention further provides for analysis of the altered gene expression levels in normal or noncancerous cells as an indicator of disease presence, positioning, prognosis, staging and grading.

According to one embodiment of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein differential expression of the genes is indicative of cancer.

In another aspect of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein increased expression of the genes that are up-regulated is indicative of cancer.

In another aspect of the invention, a method is provided for diagnosing the presence or absence of cancer in a patient. The method comprises the steps of: detecting the level of gene expression in anatomically normal or noncancerous cells in a cytology specimen consisting of cells that had previously resided in the proximity of cancer; wherein increased expression of the genes that are down-regulated is indicative of cancer.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein the differential expression of the genes is indicative of the cancer progression.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein increased expression of the genes that are down-regulated is indicative of cancer progression.

Another aspect of the invention is a method of detecting the progression of cancer in a patient. The method comprises the steps of: detecting the level of gene expression in anatomically normal or noncancerous cells in a cytology specimen consisting of cells that had previously resided in the proximity of cancer; wherein increased expression of the genes that are up-regulated is indicative of cancer progression.

The invention also includes methods of differentiating metastatic cancer from nonmetastatic cancer in a patient comprising the step of detecting the level of expression in anatomically normal or noncancerous cells in a cytology specimen consisting of cells that had previously resided in the proximity of cancer; wherein altered patterns of gene expression is indicative of metastatic cancer rather than nonmetastatic cancer.

In another aspect of the invention, a method is provided for diagnosing the presence or absence of cancer in a patient can be used to detect, for example, prostate cancer, bladder cancer, liver cancer, lung cancer, and breast cancer, just to name a few examples.

In one embodiment of the present invention the tumor is a gastrointestinal tumor, for example a tumor of the esophagus, the stomach, the pancreas, the bile tree, the liver, the small intestine, the colon or the rectum. In another embodiment the tumor is for example cancer of the esophagus, gastric cancer, cancer of the gallbladder, the pancreas, the liver, the small intestine, the colon or the rectum. The tumors according to the present invention may comprise tumors, which show detectable lymph-node involvement (node positive tumors) as well as tumors, without detectable spread to lymphnodes (node negative tumors). In one preferred embodiment of the invention the gastrointestinal tumors are tumors without detectable spread to lymph nodes.

Poor clinical outcome can be measured, for example, in terms of shortened survival or increased risk of cancer recurrence, e.g. following surgical removal of the cancer.

In another embodiment, the invention concerns a method of predicting the likelihood of the recurrence of cancer, following treatment, in a cancer patient, comprising determining the expression level of certain genes, or its expression product, in a cancer tissue obtained from the patient, normalized against a control gene or genes, and compared to the amount found in a reference cancer tissue set, wherein an expression level indicates decreased risk of recurrence following treatment.

All types of cancer are included, such as, for example, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer. The foregoing methods are particularly suitable for prognosis/classification of breast cancer.

In all previous aspects, in a specific embodiment, the expression level is determined using RNA obtained from a formalin-fixed, paraffin-embedded tissue sample. While all techniques of gene expression profiling, as well as proteomics techniques, are suitable for use in performing the foregoing aspects of the invention, the gene expression levels are often determined by reverse transcription polymerase chain reaction (RT-PCR).

The expression data can be further subjected to multivariate analysis, for example using the Cox Proportional Hazards model.

The invention further includes computer systems comprising a database containing information identifying the expression level in an identified tissue of a set of genes; and a user interface to view the information. The database may further include sequence information for the genes, information identifying the expression level for the set of genes in normal tissue and malignant tissue (metastatic and nonmetastatic) and may contain links to external databases such as GenBank.

Lastly, the invention includes methods of using the databases, such as methods of using the disclosed computer systems to present information identifying the expression level in a tissue or cell, comprising the step of comparing the expression level in the tissue or cell to the level of expression of the gene in the database.

In one embodiment, the invention is used to detect cancer by examining cytology specimens of normal (non-cancerous) cells for the differential expression of genes occurring as a consequence of these normal cells having been in the proximity of cancer cells.

In another embodiment, the invention is also used to detect cancer by examining cytology specimens of normal (non-cancerous) cells for the increased expression of the genes that are up-regulated as a consequence of these normal cells being in the proximity of cancer cells.

In another embodiment, the invention is also to detect cancer by examining cytology specimens of normal (non-cancerous) cells for the increased expression of the genes that are down-regulated as a consequence of these normal cells being in the proximity of cancer cells.

In another embodiment, the reference gene expression profile is contained within a database. In another embodiment, the comparing of profiles is carried out using a computer algorithm. In another embodiment, the method further comprises isolating the cell of the sample from the patient. In another embodiment, the method further comprises preparing the patient's gene expression profile. In another embodiment, the method further comprises: (c) providing a gene expression profile of a cell from the patient after the patient has undergone a treatment regimen for a disease state; and (d) comparing the post-treatment patient gene expression profile to the reference gene expression profile, to monitor the patient's response to the treatment regimen.

In another embodiment, the method provides for diagnosing a cancer disease state in a patient, the method comprising: (a) providing a gene expression profile of a patient's isolated cells wherein the isolated cells simultaneously express a plurality of genes at the protein level that are markers for specific for a disease; and (b) comparing the patient's gene expression profile to a reference gene expression profile obtained from a normal cell, wherein the reference gene expression profile comprises an expression value of a target gene, wherein differential expression of the target gene is indicative of a disease state, and wherein the disease state is a proliferative or hyperproliferative disorder. In one embodiment, the proliferative or hyperproliferative disorder is cancer.

In one embodiment, the reference gene expression profile comprises an expression value of a target gene selected from the group consisting of human mRNA for one or more biomarker.

In one embodiment, an alteration in the level of the one or more biomarkers as compared to control indicates colorectal cancer or pre-malignant colorectal cancer state. In one embodiment, the one or more biomarkers are chosen from a nucleic acid, a DNA, a RNA, and a protein. In one embodiment, the alteration in the level of one or more biomarkers results in an increase in the level of the mRNA. In one embodiment, the alteration in the level of one or more biomarkers results in an increase in the level of the protein. In one embodiment, the sample is isolated from cells obtained by biopsy or any other method of extraction.

In one embodiment, the e determining the level comprises analyzing the sample for the level of DNA or RNA. In one embodiment, the determining the level is carried out by (1) PCR amplification, SDA amplification, or any other method of nucleic acid amplification, (2) using a nucleic acid microarray, (3) gel electrophoresis, (4) transfer to a membrane and hybridization with a specific probe, and (5) diagnostic imaging. In one embodiment, the determining the level comprises analyzing the sample for the level of the protein. In one embodiment, the analysis is carried out by (1) incubation with a specific antibody, (2) Western blot, (3) immunohistochemistry, (4) gel electrophoresis, (5) microarray, (6) ELISA, and (7) diagnostic imaging.

In one embodiment, the variation in the expression levels of the gene or genes is used to predict the progression of the colorectal cancer or of a premalignant condition thereof, for predicting the risk of recurrence, and/or determining the type of therapy.

In one embodiment, the level of gene expression is determined by using probes. In one embodiment, the probes are antibodies. In one embodiment, the antibodies are monoclonal. In one embodiment, the expression levels are determined by immunohistochemical staining of the biological sample.

In one embodiment, the expression profile is determined by quantifying a level of expression of one or more specific cellular protein.

In one embodiment, the specific cellular proteins are either involved in a biological pathway, belong to a group of proteins with identical or similar biological function, are expressed in a stage of cell cycle, expressed in a cell type, expressed in a tissue type, expressed in an organ type, or expressed in a developmental stage, proteins whose expression and/or activity is altered in a disease or disorder type or stage, or proteins whose expression, activity or a combination thereof is altered by a drug or other treatment.

In one embodiment, the specific cellular proteins comprise at least one transcription factor. In one embodiment, the specific cellular proteins comprise at least one protein from the serine/threonine kinase family: MEK MKK3 PAK PAK6 CDK CDK8-LIKE MKK6 PAK4 CDK8 MKK4 PAK5 CDK9 MKK7 PAK3 NKIAMRE MEK5 PAK1 KKIALRE MEK2 PAK2 KKIAMRE MEK1 CaMK CASK STK9 MEKK TPL2 CAMKIIb CDK3 MEKK6 CAMKg CDK2 ASK1 CAMK2A CDC2 MEKK2 CAMKIId CDK5 MEKK3 CAMKIId-Like CDK4 MEKK4 AMPK1-Like CDK6 MEKK1 AMPKA1 CCRK CK1 CK1e PRKK-Like CDK7/CAK1 CK1d SNF1-Like CDK10 CK1a_Like STK29 PITSLRE CK1a MARK MAPK p38 CK1g1 MARK3 p38b/SAPK2 CK1g2 EMK p38g/SAPK3 CK1g3 CHK1 p38d/SAPK4 PIM2 ERK5 PIM1 ERK2 HUNK ERK1 STK33 ERK4 PKC-u ERK3 PKC-mu JNK2 PKD2 JNK1 CHK2 JNK3 DMPK WNK1 DYRK4 CDC42BPB WNK3 LCK2 DMPK-LIKE WNK4 STK9 ROCK1 STK2 MOK ROCK2 NEK3 CDK8 KPM NEK6 MSSK1 WARTS MINK1-2 SRPK1 SAST SPAK GLK PKN IRE1 TLK1 PDK1 IRE1-B TLK2 STK33 PRKR PLK TSF1 TPL2 SAK GAK WEE1-LIKE PIM1 IKKe TOPK PIM2 RAF1 HIPK2 CAMKK RAF1-LIKE YAK1 AMPKa1 RIPK1 GSK3b SNF- LIKE2 RIPK3 DYRK1A MELK TAK1 DYRK1B EMK TESK2 DYRK2 MYLK TESK1 DYRK3 DRAK1.

In one embodiment, the specific cellular proteins comprise at least one protein from the tyrosine kinase family: JAK JAK1 ABL ARG JAK2 ABL JAK3 TEC ITK TYK2 TEC SYK ZAP70 BTK SYK TXK SRC LYN BMX HCK FES FER BLK FES LCK ACK TNK1 FYN ACK1 FGR FAK FAK SRC PYK2 YES1 MATK CSK FRK B. Receptor tyrosine kinases: DDR1 RET ERBB4 EPHA3 LMR2 DDR2 TIE ERBB2 EPHA5 LMR1 ROS TEK ERBB3 EPHA4 AXL FLT3 EGFR EPHA7 MER FGFR4 EPHA8 SKY FGFR3 EPHB 1 MET FGFR2 EPHB2 RON FGFR1 EPHB3 RYK VEGFR1 EPHB4 TRKA VEGFR2 EPHB6 TRKB VEGFR3 EPHA1 TRKC KIT EPHA2 MUSK CSF1R INSR CCK4 PDGFRA/B IGF1R ALK INSRR LTK ROR1/2.

In one embodiment, the specific cellular proteins comprise at least one transcription factor: ERK 1/2 Elk-1, Stat 1/3, Ets-1, ER, c-Myc, SRF, CREB SAPK/JNK c-Jun, ATF-2, Elk-1, p53, DPC4 p38 MAPK ATF-2, MEF2C, Elk-1, Myc/Max, Statl, CREB, CHOP ERK5/BMK MEF2C p90.sup.rsk c-Fos, SRF, CREB MSK1 CREB JAKs STATs PKA CREB, SAF-1, GATA-4, SOX9, HNF-4, AR PKB/Akt forkhead, AFX GSK3.beta. AP1, beta-catenin, C/EBPalpha, CREB, HSF-1, Myc, NFAT, NF.kappa.B ALKs SMADs CaMK Ets-1, CREB, STAT1.

In one embodiment, the specific cellular protein is selected from the group consisting of: Cyclin A, Cyclin B, Cyclin D1, Cyclin D3, Cyclin E, CDK1, CDK2, CDK4, CDK6, E2F, CDC2, cdc25c, Cdc25A, Chk2, Chk1, pRb, p53, p21, p27, and Wee1.

In one embodiment, the expression level is determined by determining the levels of a marker protein. In one embodiment, the marker protein is selected from the group consisting of p16^{INK4a}, HPV E6, HPV E7, HPV E2 HPV E4, HPV L1, HPV L2, p27, p21, p15, p19, p53, pRb, and MDM2.

In one embodiment, the disease is a cell proliferative disorder, cancer or a precursor lesion. In another embodiment, the cancer is cancer of the head and the neck, cancer of the respiratory tract, cancer of the gastrointestinal tract, cancer of the skin and its appendages, cancer of the central and peripheral nervous system, cancer of the urinary system, cancer of the reproductive system, anogenital cancer, cancer of the endocrine system, cancer of the soft tissues and bone, or cancer of the lymphopoietic and hematopoietic system.

In one embodiment, the reference gene expression profile comprises expression values of human mRNA for tyrosine phosphatase, X-linked mental retardation candidate gene, human interleukin-10 receptor mRNA, H-interferon inducible peptide, interferon-inducible 56 kD protein, insulin-like growth factor binding protein 5, winged helix transcription factor, interferon gamma, human mRNA zinc finger protein, apolipoprotein D precursor, a tumor suppressor protein or antigen, Homo sapiens Kueppel family zinc finger protein, major group rhinovirus receptor precursor, ICE like protease, caspase-10/b, and human transcription factor SIM2.

In one embodiment, the invention uses the gene expression profile of at least one tumor-suppressor gene such as the p53 gene, the Rb gene, etc. In one embodiment, the tumor-suppressor gene is the Casp7 gene. In another embodiment, the tumor-suppressor gene is the Dcc gene. In another embodiment, the tumor-suppressor gene is the p53 gene. In another embodiment, the tumor-suppressor gene is one of several known tumor-suppressor genes, such as (Rb1, Wt1, Nf1, Nf2, Apc, Tsc1, Tsc2, Dpc4, Brca1, Brca2, Pten, Lkb1, Msh2, Mlh1, Cdh1, Vh1, Cdkn2a, Ptch, Men1, E2f1, Chek2, Cdkn1a, Smarcb1, Braf, Kit, Ret, Casp3, Egfr, Jun, Gstm1, Gstt1, Mthfr, Gstp1, Cypla1, Xrcc1, Ercc2, Nat2, Tnf Il1b, Il10, and Ar).

In another embodiment, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

In another embodiment, the detecting the expression of one or more marker that is specific for more than one proliferative disease comprises detecting the presence, absence, abundance and/or expression of physiological, genetic and/or cellular expression and/or cell count, preferably the detecting the expression comprises detecting the expression of protein, mRNA expression and/or the presence or absence of DNA methylation in one or more of the markers. In another embodiment, the detecting the expression of protein comprises marker-specific antibodies, ELISA, cell sorting techniques, Western blot, or the detection of labeled protein, and the measuring the mRNA expression comprises detection of labeled mRNA or Northern blot.

In another embodiment, the determination of cancer comprises determining a chance of disease-free survival, and/or monitoring disease progression in the subject. In another embodiment, the determination of cancer comprises determining metastatic disease by identifying tissue markers in the sample that are foreign to the tissue from which the sample is taken from. In another embodiment, the proliferative disease is in the early pre-clinical stage exhibiting no clinical symptoms.

Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247 283 (1999)); RNAse protection assays (Hod, Biotechniques 13:852 854 (1992)); and reverse transcription polymerase chain reaction (RT-PCR) (Weis et al., Trends in Genetics 8:263 264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Reverse Transcriptase PCR (RT-PCR)

Of the techniques listed above, the most sensitive and most flexible, quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

In another embodiment, the first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan RT-PCR can be performed using commercially available equipment, such as, for example, ABI PRISM 7700 Sequence Detection System (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and .beta.-actin.

A more recent variation of the RT-PCR technique is the real time quantitative PCR, which measures PCR product accumulation through a dual-labeled fluorigenic probe (i.e., TaqMan probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996).

Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

In papers published by Yu et al (2004, J Clin Oncol 22:2790-2799.) and Chandran et al (2005, BMC Cancer. 13; 5:45), it was disclosed that many hundreds of genes are differentially expressed in prostate cancer compared to their expression patterns in tissue from normal prostates. Surprisingly, it was also shown that most of these differentially expressed genes are also differentially expressed in non-cancerous (normal) tissue that is adjacent to or near prostate tumors. Thus, detection of these cancers induced differential gene expression patterns in normal cells can indicate the presence of cancer that was not sampled in a needle core biopsy. Detection of cancers through this indirect means by detection of cancer induced differential gene expression in normal cells will increase the sensitivity of biopsies to detect cancer.

In another embodiment, the invention is applied to prostate cancer detection, it would be possible to detect cancer that was unsampled in a needle core biopsy by searching the entire length of the biopsy core for differential gene expression using immunohistchemistry. This is not the preferred method to detect cancer induced differential gene expression because of the time consuming nature of such a search. In addition, such a search would need to be performed on all needle core biopsies taken from a patient. Most patients are sampled with ten or twelve needle core biopsies. The need to make lengthwise searches of ten or twelve biopsy cores per patient would be highly problematic due to the large human resources costs associated with this kind of search. Instead, we propose to search all of a patient's needle core biopsies in a single cytology specimen that is derived from the needle core biopsies.

This cytology specimen is created when the needle core biopsies are fixed (such as with formalin) or possibly rinsed in an aqueous solution. In one embodiment, the solution is an aqueous buffer about pH 6 to about pH 11, preferably an aqueous buffer about pH 7 to about pH 10, such as for example Phosphate Buffered Saline at pH 7.4. Fixation occurs in jars that are also used to transport specimens to pathology reference labs for further processing and evaluation. Interestingly and importantly, when the needle core biopsies are placed in the fixative (and possibly other solutions as well) many cells detach from the biopsy core and become suspended in the fixative. These loose cells in the fixative are a sampling of the cells in the needle core biopsy. If all (or some) of the fixative from all (or some) of the fixative jars were pooled, then a cytology specimen would be created that consisted of a sampling of all (or some portion) of the needle core biopsies. Cells from this cytology specimen could be placed on a microscope slide and stained with an immunohistochemical (or an immunofluorescent or a molecular probe) reagent that could indicate the presence in a cell of a protein (or RNA) that was up-regulated or down-regulated due to the proximity of the cell in the prostate gland to a prostate tumor.

Each field of view on such a microscope slide will contain many cells or clumps of cells each derived from a different part of one the various needle core biopsies from which the pooled cytology specimen was derived. Thus, by scanning one or a small number of fields on the microscope slide, one can quickly examine cells sampled from all portions of all needle core biopsies. If cells are identified that express one or more genes differentially in a pattern indicative of cancer, then the presence of prostate cancer is indicated even if no cancer is present in the tissue actually sampled by the needle core biopsies. Examples of some of the genes that might be used to predict unseen cancer are AMACR (alpha-Methylacyl coenzyme A racemase), Hepsin (TMPRSS1), c-fos, and c-jun. Other genes are suggested by the work of Yu et al., (2004) and Chandran et al (2005).

While not wishing to be bound by theory, it is expected that there are many, perhaps hundreds, of other genes that could be used to detect cancer in the current invention. One embodiment of the current invention is the use of the cells, that detach from needle core prostate biopsy specimens when they are placed in fixative solution, as a cytology specimen to detect gene expression patterns in non-cancerous cells that are indicative of cancer that may not be directly observable in needle core biopsies from which this cytology specimen was derived.

In another embodiment, the sample according to the method of the present invention is any sample of cells or body fluids containing cellular components. Such samples may be for example gastrointestinal secretions, stool, bile, biopsies, cell- and tissue-samples. Biopsies as used in the context of the present invention may comprise e.g. resection samples of tumors, tissue samples prepared by endoscopic means or needle biopsies of organs. Furthermore any sample potentially containing the nucleotides to be detected may be a sample according to the present invention. Such samples may comprise for example intact cells, lysed cells or any liquids containing proteins, peptides or nucleic acids. Even solids, to which cells, cell fragments or marker molecules may adhere, may be samples according to the present invention. Such solids may comprise for example membranes, glass slides, beads etc.

In another embodiment, the preparation of a sample may comprise e.g. obtaining a sample of a tissue, a body fluid, of cells, of cell debris from a patient. According to the present invention preparation of the sample may also comprise several steps of further preparations of the sample, such as preparation of dissections, preparation of lysed cells, preparation of tissue arrays, isolation of polypeptides or nucleic acids, preparation of solid phase fixed peptides or nucleic acids or preparation of beads, membranes or slides to which the molecules to be determined are coupled covalently or non-covalently.

A further embodiment of the current invention would be to examine expression patterns of more than one gene, including possibly both differentially expressed and non-differentially (constitutively) expressed genes, and combining the information from these various expression patterns in an analysis using a statistical algorithm to provide a better estimate of the probability of unseen cancer. This is a multivariate as opposed to a univariate analysis of the gene expression data.

Another embodiment of the invention is the use of the cells as a cytology specimen to detect gene expression patterns in non-cancerous cells that are indicative of the cancer progression that may not be directly observable in needle core biopsies from which this cytology specimen was derived. The increased expression of the genes maybe down-regulated or up-regulated as indicative of cancer progression.

Another embodiment of the invention is to examine gene expression patterns of one or more genes of metastatic cancer to detect the level of expression in anatomically normal or noncancerous cells in a cytology specimen as indicative of metastatic cancer or nonmetastatic cancer.

Another embodiment of the invention would be to examine in morphologically benign cells the expression levels of genes known to be differentially expressed as a result of having been in the proximity of cancer. Examples of such examined differentially expressed genes may include, among others, one or more of the following genes: FGFR4, c-FOS, BTG2, IER2, c-Jun, JunD, JunB, CYR61, CyclinD1, EGR1, AMACR, Duffy Blood Group antigen and CD163. In addition, one or more genes could be examined that are known not to be differentially expressed or only minimally differentially expressed. Examples of such non-differentially expressed genes that may be examined include, among other possible genes: HPRT1, ALAS1, Tubulin-a, GAPDH and the androgen receptor. In this embodiment, the expression levels of examined genes may be evaluated individually.

In another embodiment, the expression levels of some or all of the examined genes (both differentially expressed genes and/or non-differentially expressed genes) may be compared to each other generating an expression level ratio. These expression level ratios and/or individual gene expression levels would comprise the input variables that would be analyzed by a statistical algorithm the output of which would be the probability of unseen or unsampled cancer or prognostic and/or predictive information about a patient's cancer (whether directly observed or not). This statistical algorithm may be derived from any of a variety of statistical methods such as but not limited to logistical regression, artificial neural networks, principle component analysis, support vector machines or other methods.

In another embodiment, the method is also applicable to the detection of bladder cancer, its progression level and whether it is metastatic or nonmetastatic cancer. Every year, tens of thousands of patients are diagnosed with bladder cancer. In addition, there are hundreds of thousands of patients who have been previously diagnosed with and treated for bladder cancer. These previously diagnoses and treated patients are monitored for recurrence of their disease.

An important part of the current practice for diagnosing and monitoring of bladder cancer patients involve examinations by pathologists of cytology specimens. These cytology specimens are loose cells derived from the lining of the bladder or other parts of the urinary system and are collected in either urine or bladder lavages. Pathologists examine bladder cytology specimens for the presence of cancer cells. Large numbers of noncancerous cells may also be present in the cytology specimen. Identifying bladder cancer with a cytology specimen can be problematic due to imperfect sensitivity and specificity.

In another embodiment, the means to increase the performance of bladder cytology is to examine the noncancerous cells in the cytology specimen for cancer specific gene expression patterns that arose in these cells due to their proximity to cancer cells when they were embedded in the lining of the urinary system. As with the description of the invention as it is applied to the detection of prostate cancer, this analysis of the expression patterns in bladder cytologies can be performed using immunohistochemical, immunofluorescent or a molecular probe reagent(s) that can be analyzed by either a univariate or multivariate method using a statistical algorithm.

In another embodiment, the method can also apply to the detection, progression level and differentiation of metastatic from nonmetastatic breast cancer. Fine needle aspirate (FNA) biopsy specimens of the breast can be used to detect breast cancer. This is a cytology specimen that is examined by a pathologist in an attempt to detect cancer cells. Similarly to the applications described for prostate and bladder cancer, this method suffers from imperfect sensitivity and specificity.

The performance of FNA could be improved by including an analysis for cancer specific alterations in gene expression patterns that occur in noncancerous cells resided in proximity to cancer cells in the breast. Just as with the previous two examples, this analysis of the expression patterns in FNAs can be performed using immunohistochemical or immunoperoxidase, immunofluorescent or a molecular probe reagent(s) that can be analyzed by either a univariate or multivariate method using a statistical algorithm.

In another embodiment, breast cancer can also be diagnosed with needle core biopsies. It is envisioned that a cytology specimen could be created from a breast needle core biopsy in a manner identical to that for a prostate needle core biopsy described previously. Normal or non-cancerous cells from such cytology specimens could also be examined for indications of cancer just as described for the prostate specimens.

The methods of the present invention are not limited to prostate, bladder or breast cancer. In addition to the applications previously described, the current invention can be used to improve the detection of cancer in any directly obtained cytology specimen or cytology specimens resulting from cell dispersion into fixative solutions or other solutions. The key to all applications of the current invention is to examine the normal and/or noncancerous cells in such cytology specimens to identify altered patterns of gene expression that arose in these cells as a result of their proximity to cancer in the organ from which the cytology specimen was derived. One can easily imagine applications of the current invention to the detection of cancers of the lung, stomach, intestinal tract, brain, kidney testis thyroid, lymph node or blood.

1. A method for diagnosis, or monitoring the occurrence, development, progression or treatment, of a proliferative disorder, the method comprising the steps of (a) obtaining a biological sample comprising normal or noncancerous cells from the subject, (b) determining whether the cells in the sample have altered patterns of gene expression as compared to one or more reference control cells, wherein the altered patterns of gene expression arose as a consequence of proximity to cancer cells and wherein the result of the comparison is indicative of the presence, absence, occurrence, development, progression, or effectiveness of treatment of the disorder in the subject.

2. The method of claim 1, wherein the determining is made using a cytology specimen comprising normal or noncancerous cells that had previously resided in the proximity of cancer cells;

3. The method of claim 1, wherein the proliferative disease is cancer.

4. The method of claim 1, wherein the determining is made using an immunohistochemical reagent.

5. The method of claim 1, wherein the determining is made using an immunofluorescent reagent.

6. The method of claim 5, wherein the reagent is a Q-dot labeled immunocomplex.

7. The method of claim 1, wherein the determining is made using a molecular probe reagent.

8. The method of claim 7, wherein the reagent is a molecular beacon, aptamer or other oligonucleotide probe.

9. The method of claim 8, wherein the oligonucleotide probe is used to direct nucleic acid amplification by any means familiar to those skilled in the art.

10. The method of claim 1, wherein more than one gene is examined for an altered pattern of gene expression.

11. The method of claim 10, wherein the data for altered patterns of gene expression from more than one gene is analyzed using a statistical algorithm.

12. The method of claim 1, wherein more than one cytology specimen are examined from a single subject.

13. The method of claim 12, wherein the comparison of the differential gene expression patterns between the cytology specimens provides information concerning the location of a cancer that was not directly sampled in the cytology specimens or from biopsy specimens from which the cytology specimens may have been derived.

14. The method of claim 2, wherein increased expression of the genes that are up-regulated is indicative of cancer.

15. The method of claim 2, wherein increased expression of the genes that are down-regulated is indicative of cancer.

16. The method of claim 2, wherein the differential expression of the genes is indicative of the cancer progression.

17. The method of claim 2, wherein increased expression of the genes that are down-regulated is indicative of cancer progression.

18. The method of claim 2, wherein increased expression of the genes that are up-regulated is indicative of cancer progression.

19. The method of claim 2, wherein altered patterns of gene expression is indicative of metastatic cancer rather than nonmetastatic cancer.

20. The method of claim 2, wherein method is provided for diagnosing the presence or absence of cancer in a patient can be used to detect, for example, prostate cancer, bladder cancer, liver cancer, lung cancer, and breast cancer.

21. The method of claim 2, wherein the gene expression levels are determined by reverse transcription polymerase chain reaction (RT-PCR).

22. The method of claim 2, wherein the expression data is further subjected to multivariate analysis.

23. A method for diagnosis, or monitoring the occurrence, development, progression or treatment, of a proliferative disorder, the method comprising the steps of (a) obtaining a biological sample comprising normal or noncancerous cells from the subject, (b) generating a gene expression profile from the sample; (c) comparing the gene expression profile to one or more reference expression profiles; (d) determining whether the cells in the sample have altered patterns of gene expression as compared to one or more reference control cells, wherein the altered patterns of gene expression arose as a consequence of proximity to cancer cells and wherein the difference or similarity between the gene expression profile and the one or more reference expression profiles is indicative of the presence, absence, occurrence, development, progression, or effectiveness of treatment of the disorder in the subject.

24. The method of claim 23, wherein the determining is made using a cytology specimen comprising normal or noncancerous cells that had previously resided in the proximity of cancer cells.

25. The method of claim 23, wherein the proliferative disease is cancer.

26. The method of claim 23, wherein the determining is made using an immunohistochemical reagent.

27. A method for diagnosis, or monitoring the occurrence, development, progression or treatment, of a cancer, the method comprising the steps of (a) obtaining a biological sample comprising normal or noncancerous cells from the subject, (b) determining whether the normal or noncancerous cells in the sample have altered patterns of gene expression; and (c) classifying a biological sample into one of three groups, the first group comprises no abnormal elevation, whereby the first group indicates the best prognosis, the second group indicates an intermediate prognosis, and the third group indicates the worst prognosis.

28. The method of claim 27, wherein the determining is made using a cytology specimen comprising normal or noncancerous cells that had previously resided in the proximity of cancer cells.

29. A method of diagnosing cancer in a subject, comprising: (a) obtaining a biological sample comprising normal or noncancerous cells from the subject, (b) detecting a level of expression in the sample of selected marker genes by contacting the nucleic acid capture probes with nucleic acids from the cell sample so as to allow for the hybridization of the nucleic acid capture probes with the nucleic acids from the cell sample; and (c) comparing the level of expression of the selected marker genes in the cell sample to the level of expression of the same marker genes in a normal cell sample of the same tissue type, wherein the presence of a cancer cell is indicated if the level of expression of one or more of the selected marker genes in the cell sample is greater than the level of expression of the same marker genes in the normal cell sample of the same tissue type.

30. The method of claim 29 wherein the determining is made using a cytology specimen comprising normal or noncancerous cells that had previously resided in the proximity of cancer cells;

31. The method of claim 30, wherein the presence of a cancer is indicated if the level of expression of three or more selected marker genes in the cell sample is greater than the level of expression of the same marker genes in the normal cell sample of the same tissue type.

32. The method of claim 30, wherein the presence of a cancer is detected if the level of expression of seven or more selected marker genes in the cell sample is greater than the level of expression of the same marker genes in the normal cell sample of the same tissue type.

33. The method of claim 30, wherein the step of comparing the level of expression of the selected marker genes further comprises using a class prediction algorithm to differentiate the level of expression of the selected marker genes in the cell sample from the level of expression of the same marker genes in the normal cell sample of the same tissue type.

34. The method of claim 33, wherein the one or more class prediction algorithms is selected from the group consisting of compound covariate predictor, diagonal linear discriminant analysis, nearest neighbor predictor, nearest centroid predictor, and support vector machine predictor.

35. The method of claim 30, wherein the presence of a cancer cell is indicated if the level of expression in the cell sample of at least one of the selected marker genes is at least two times the level of expression of the same marker gene in the normal cell sample of the same tissue type.

36. The method of claim 30, wherein the presence of a cancer cell is indicated if the level of expression in the cell sample of at least one of the selected marker genes is at least three times the level of expression of the same marker gene in the normal cell sample of the same tissue type.

37. The method of claim 30, wherein the presence of cancer being detected is selected from the group consisting of ovarian carcinoma, serous adenocarcinoma, clear cell adenocarcinoma, endometrioid carcinoma, mucinous adenocarcinoma, breast adenocarcinoma, and infiltrating ductal carcinoma.

38. The method of claim 37, wherein comparing the level of expression of the selected marker genes further comprises using a class prediction algorithm to differentiate the level of expression of the selected marker genes in the cell sample from the level of expression of the same marker genes in the normal cell sample of the same tissue type.

39. The method of claim 38, wherein comparing the level of expression of the selected marker genes further comprises differentiating the level of expression of the selected marker genes in the cell sample from the level of expression of the same marker genes in the normal cell sample of the same tissue type using one or more class prediction algorithms selected from the group consisting of compound covariate predictor, diagonal linear discriminant analysis, nearest neighbor predictor, nearest centroid predictor, and support vector machine predictor.

40. The method of claim 39, wherein the level of expression of marker genes is determined using RT-PCR, PCR, nucleic acid blotting, dot blotting, or microarray.

41. A method according to claim 40, wherein the level of gene expression is determined by using probes.

42. A method for diagnosing a proliferative disease in a subject comprising: (a) obtaining a cytology sample comprising normal or noncancerous cells from the subject wherein the normal or noncancerous cells had previously resided in the proximity of cancer cells, (b) detecting the presence, absence, abundance and/or expression of one or more markers and determining therefrom upon the presence or absence of a proliferative disease; and (c) detecting the presence, absence, abundance and/or expression of one or more cell- and/or tissue-markers and determining therefrom if the one or more cell- and/or tissue-markers are atypically present, absent or present at above normal levels within the sample; and (d) determining the presence or absence of a cell proliferative disorder and location thereof based on the presence, absence, abundance and/or expression as detected in step b) and c).

43. The method according to claim 42, further comprising detecting the presence, absence, abundance and/or expression of one or more markers and determining therefrom characteristics of the cell proliferative disorder.

44. The method according to claim 43, wherein the marker in step b) is indicative of more than one proliferative disease.

45. The method according to claim44, wherein the proliferative disease is cancer.

46. The method according to claim 44, wherein the detecting the presence, absence, abundance and/or expression of one or more markers comprises detecting physiological, genetic, and/or cellular presence, absence, abundance and/or expression, and cell count.

47. The method according to claim 46, wherein the detecting the expression comprises detecting the expression of protein, mRNA expression and/or the presence or absence of DNA methylation in one or more of the markers.

48. The method according to any of claims 47, wherein the characterizing cancer comprises determining the likelihood of disease-free survival, and/or monitoring disease progression in the subject.

49. The method according to claim 48, wherein the characterizing cancer comprises determining metastatic disease.

50. The method according to claim 49, wherein the characterizing cancer comprises determining relapse of the disease after complete resection of the tumor in the subject by identifying tissue markers and cancer markers in the sample that are identical to the removed tumor.
